(19) United States
(12) Patent Application Publication
(10) Pub. No.: US 2019/0100577 A1
(43) Pub. Date: Apr. 4, 2019

(54) SELECTIVELY AND FULLY CLEAVABLE FLUORESCENT PROBES FOR SEQUENTIAL, ITRATIVE IMMUNOSTAINING

(71) Applicant: The Trustees of Columbia University in the City of New York, New York, NY (US)

(72) Inventors: Harshad D. Vishwasrao, Bethesda, MD (US); Eric R. Kandel, New York, NY (US)

(73) Assignee: The Trustees of Columbia University in the City of New York, New York, NY (US)

(21) Appl. No.: 16/087,567

(22) PCT Filed: Mar. 24, 2017
(86) PCT No.: PCT/US17/23985

§ 371 Date: Sep. 21, 2018

Related U.S. Application Data
(60) Provisional application No. 62/313,027, filed on Mar. 24, 2016.

(51) Int. Cl. C07K 16/18; G01N 33/58

(52) U.S. Cl. CPC C07K 16/18; G01N 33/6857; G01N 33/582

(57) ABSTRACT

The subject invention provides a selectively cleavable probe comprising an F(ab) fragment linked to one or more labels by a chemically cleavable disulfide bond.

The subject invention also provides a process of immunostaining a sample comprising staining the sample with a selectively cleavable probe.

The subject invention further provides a process of immunostaining a sample comprising (a) obtaining a probe comprising a primary antibody against a preselected target linked to biotin, (b) staining the sample with the probe of step (a), and (c) staining the sample with a selectively cleavable probe, wherein the F(ab) fragment is an anti-biotin F(ab) fragment.

This application claims the benefit of U.S. Provisional Application No. 62/313,027, filed Mar. 24, 2016, the entire contents of which are hereby incorporated by reference herein.

Throughout this application various publications are referenced, most typically by the last name of the first author and the year of publication. Full citations for these publications are set forth in a section entitled References immediately preceding the claims. The disclosures of these publications in their entireties are hereby incorporated by reference into this application in order to more fully describe the state of the art to which the invention relates.

### BACKGROUND OF INVENTION

Every function of neurons, including sensation, action, cognition, learning, and memory, involves highly coordinated interactions among large networks of proteins. Proteomic studies of both pre-synaptic and post-synaptic compartments have enumerated hundreds of different proteins that are organized into clustered interaction networks (Abul-Husn 2009, Li 2004, Choudhary 2006, Yoshimura 2004, Collins 2006, Husi 2000, Pocklington 2006). With the emergence of systems biology and proteomics, it has become increasingly clear that developing robust, predictive models of the molecular mechanisms of cellular functions such as learning and memory will require an understanding of how these networks of molecular interactions function. Molecular interaction networks are becoming increasingly central to drug design. Systems level phenomena such as cross talk between signaling pathways and ‘off-target’ drug interactions lead to adverse side effects and poor drug efficacy, together accounting for 30% of all drug failures in clinical trials (Hopkins, 2008). From the perspective of both basic and translational neuroscience, the systems level analysis of molecular interaction networks is an emerging paradigm that promises to be transformative.

Experimental approaches to detecting large sets of molecular interactions in cells have been limited. Biochemical methods for detecting protein-protein interactions, such as co-immunoprecipitation, are able to detect large numbers of interactions, enabling the reconstruction of complex interaction networks. However, these approaches typically involve homogenizing a large volume of the cell/tissue sample, thereby losing a great deal of information about the spatial variation of protein interaction networks. This spatial variation is particularly important in highly heterogeneous tissues such as the brain, where interaction networks are expected to vary not only from cell type to cell type (e.g neurons versus glia) but also from compartment to compartment within the same cell (e.g. dendritic spine versus dendritic shaft).

The ability to stain a single fixed cell or tissue sample with many different fluorescent probes for many different biomolecules i) maximizes the information that can be gained from a single cell/tissue sample and ii) reveals relationships between the spatial distributions of these biomolecules that might not have been evident had they been probed individually on many different samples. Because of the advantages of single sample staining, several approaches have been developed to stain a sample with multiple probes either simultaneously or sequentially. These approaches are typically (though not always) collectively called ‘multiplex imaging’ (Gerdes et al., 2013; Jungmann et al., 2014; Micheva & Smith, 2007; Nelson et al., 2013; Schubert et al., 2006). Multiplex imaging holds tremendous potential to elucidate how large sets of proteins are spatially coordinated and therefore how molecular interaction networks vary spatially.

The present invention provides several advantages over existing multiplex imaging approaches.

### SUMMARY OF THE INVENTION

The subject invention provides a selectively cleavable probe comprising an F(ab) fragment linked to one or more labels by a chemically cleavable disulfide bond.

The subject invention also provides a process of immunostaining a sample comprising staining the sample with a selectively cleavable probe.

The subject invention further provides a process of immunostaining a sample comprising
a) obtaining a probe comprising a primary antibody against a preselected target linked to biotin,
b) staining the sample with the probe of step a),
c) staining the sample with a selectively cleavable probe, wherein the F(ab) fragment is an anti-biotin F(ab) fragment.

### BRIEF DESCRIPTION OF THE FIGURES

FIG. 1. Cleavable staining of a sample is accomplished in two steps. (A) A suitable primary antibody against an antigen in the sample is first modified by coupling it to biotin via a cleavable disulfide bond. This reaction between the primary antibody and NHS-SS-biotin is rapid (15 minutes, room temperature). The cleavably biotinylated primary is then applied to the sample where it binds the target antigen. (B) Anti-biotin F(ab)-SS-GFP probes are then added to the sample to bind the biotin on the primary antibody. The antigen is now fluorescently labeled and can be imaged. (C) To destain the sample, a disulfide reducing agent is applied to the sample to cleave the disulfide bonds, leaving the primary antibody stripped of GFP and accessible biotin moieties. Some residual non-fluorescent F(ab) may remain (black arrow) due to steric protection of the disulfide bond by the binding F(ab). The F(ab)-SS-XFP probe (D) consists of an anti-biotin F(ab) fragment that has been linked to one or more molecules of GFP or a similar fluorescent protein via a cleavable disulfide bond.

FIG. 2. Fluorescence immunostain overlaid on DIC. Cleavably biotinylated primary antibodies and anti-biotin F(ab)-SS-GFP yield immunostains (right column) that are comparable to those generated by conventional primary and secondary antibodies (left column). Conventional immunostains were done using unmodified primary antibodies and species specific secondary antibodies labeled with Alexa 647. Both types of stains exhibit the intercellular adhesion ridges characteristic of α N-Catenin (arrows), the lamellar enrichment characteristic of Arp 2/3 (arrows), and the stress fiber localization of Myosin 2b (arrows).

FIG. 3. Cleavable stains can be destained by disulfide reducing agents such as TCEP in a time and concentration dependent manner. COS-7 cells cleavably stained for Myosin 2b (left column) show substantial or complete destaining with a one hour wash in TCEP buffer (TCEP in 100 mM Tris-HCl, 150 mM NaCl, 0.3% Triton X-100, pH 7.4). The level of destaining (right column, contrast enhanced 10× to reveal residual stain) was found to be complete at 20 mM TCEP.

FIG. 4. Illustration of iterative serial stain protocol. (A) The sample is first stained with any required permanent labels. In this case f-actin is labeled with Phalloidin-Alexa 647 (B) The first actin binding protein (represented by a square) is then stained using a cleavably biotinylated primary antibody and an anti-biotin F(ab)-SS-GFP applied in two sequential incubations. The permanent and cleavable fluorescence stains are both imaged. (C) After imaging, the GFP on the F(ab) is cleaved with TCEP, leaving the permanent stain unaffected. (D) The next actin binding protein (represented by a triangle) is then stained with a cleavably labeled primary antibody and an anti-biotin F(ab)-SS-GFP and the process is repeated.

FIG. 5. A practical illustration of the iterative serial stain. Fixed COS-7 cells were stained with a conventional unmodified antibody against β-Actin and a species specific secondary antibody labeled with Alexa 647 (right column). Multiple proteins of interest (α N-Catenin (Round 1), Arp 2/3 (Round 2), Myosin 2b (Round 3), and Drebrin A/E (Round 4)) were then stained, imaged, and destained in sequential rounds (left column). The permanent stain remained largely unaffected.

FIG. 6. Excitation induced formation of a non-cleavable stain. COS-7 cells are stained using an anti-Myosin2b primary antibody and an anti-Rabbit Fab-SS-CF488A (A) or Fab-SS-GFP (C). CF488A (manufactured by Biotium) is a small fluorescent molecule similar to Fluorescein and Alexa 488. A field of view in the sample is imaged, the fluorophore on the entire sample is then cleaved off using TCEP. The same field of view in the sample, except shifted to the left by half a frame, is re-imaged after cleaving (B,D). In FIGS. 6B and 6D then, the left half-field was not imaged prior to TCEP cleaving while the right half-field was imaged prior to TCEP cleaving. CF488A (B) is rendered partially non-cleavable by imaging prior to TCEP treatment. GFP fluorescence (D) however remains fully deactivatable by TCEP after imaging. The contrast in (B) and (D) is enhanced by a factor of 20 to better show any residual stain.

FIG. 7. Anti-biotin F(ab)-SS-mCherry cleaves under similar conditions as F(ab)-SS-GFP probes, however with a slightly higher residual background. This weak residual background was found to be sample wide and was not elevated in regions that had been imaged prior to cleaving. This indicates a less than complete cleaving reaction, but no dependence on photo-excitation.

FIG. 8. (A) Cleavable bond S-S, top left: CF640R, top middle: Cy5, top right: CF594, bottom left: fluorescein, bottom middle: DyLight 650, bottom right: Atto 655. Excitation induced non-cleavability was observed for all tested small molecule dyes. In each case a region of the sample was imaged, then the dye was cleaved using TCEP, then the same region was imaged again except shifted over one half frame. This allows a side by side comparison of the residual signal in cells that had and had not been exposed to excitation light prior to cleaving.

(B) Cleavable bond N═N. Excitation induced non-cleavability was not unique to disulfide linkage of the fluorophore, nor did it require a direct covalent path from the cleavable bond to the fluorophore. In the Myosin 2B staining depicted, the F(ab) fragment was conjugated to biotin via a cleavable diazo bond. The biotin was then detected using an Alexa 488 labeled Streptavidin or anti-biotin IgG. Despite the significantly different molecular structures involved, photo-excitation still yielded a non-cleavable residual stain.

FIG. 9. F(ab) fragments against primary antibody host species (rabbit, mouse, etc.) are linked to GFP or similar fluorescent proteins (YFP, mCherry, etc.) via cleavable disulfide bonds. A primary antibody can then be bound by the appropriate F(ab)-SS-GFP against its host species, yielding a primary antibody +F(ab)-SS-GFP complex.

FIG. 10. Illustration of sequential multiplex stain protocol. In this illustration, actin filaments are labeled with a conventional permanent fluorescent label—Phalloidin-Rhodamine. Various actin binding proteins are then sequentially stained using F(ab)-SS-GFP probes. A) A primary antibody against the first actin binding protein is incubated with a F(ab)-SS-GFP against the host species of the primary. The mixture is then filtered through a 100 kD molecular weight centrifugal filter, leaving only the F(ab)-SS-GFP bound primary antibody which can then be applied to a sample. B) The sample is first stained with any required permanent labels. In this case f-actin is labeled with Phalloidin-Rhodamine. C) The F(ab)-SS-GFP labeled primary antibody against the first actin binding protein is then applied to label the first actin binding protein. The permanent and cleavable fluorescence stains are both imaged. D) After imaging, the GFP on the F(ab) is cleaved with TCEP, leaving the permanent stain unaffected. E) The next actin binding protein is then stained with a primary antibody+F(ab)-SS-GFP complex and the process is repeated.

FIG. 11. Labeled Fab fragments retain their functionality and specificity. COS-7 cells were stained with primary antibodies against Myosin 2b (A) and Tubulin (B), they were then stained with both Fab-SS-Fluorescein fragments (green) and conventional Cy3 labeled secondary antibodies. The high co-localization of the two stains indicates the functionality and specificity of Fab-SS-Fluorescein. The fluorescent label on Fab can be cleaved by TCEP. A primary IgG+Fab-SS-Fluorescein stain against Myosin 2B in COS-7 cells (C) can be almost entirely de-stained by TCEP (D). This de-staining depends on time and the concentration of TCEP, as revealed by the decrease in cellular Fluorescein fluorescence (normalized to initial intensity) upon TCEP treatment (E).

FIG. 12. The same field of view on a sample can be repeatedly stained, imaged and de-stained for several candidate interactor proteins (A; Stain Round 1-5). The stain on the f-Actin target however shows a minimal decrease (˜15%) after five rounds of interactor staining and de-staining (B). The interaction between each interactor and the target can be qualitatively estimated (C) as their co-localization (Pearson's correlation coefficient) over a region of interest (e.g. a lamella, L1; an adhesion ridge between two cells, A1; and a stress fiber, SF1).

FIG. 13. Fluorescence lifetime imaging in COS-7 cells of α-N-Catenin stained using a primary antibody+Fab-SS-Fluorescein complex in the absence of (A) and the presence of (B) a Phalloidin-CF568 FRET acceptor label on f-Actin. Adhesion ridges between adjacent cells are rich in α-N-Catenin and f-Actin, giving an average lifetime (tm) of ˜3.5 ns in the absence of an acceptor label (arrows in A) and a reduced lifetime of ˜3.1-3.3 ns in the presence of an acceptor label (arrows in B).

### DETAILED DESCRIPTION OF THE INVENTION

The subject invention provides a selectively cleavable probe comprising an F(ab) fragment linked to one or more labels by a chemically cleavable disulfide bond.

In one embodiment, the label is a fluorescent label. In another embodiment, the label is a fluorescent protein.

In an alternative embodiment, the F(ab) fragment is a monovalent F(ab) fragment from a secondary IgG antibody. In another embodiment, the F(ab) fragment is an anti-Rabbit fragment. In a further embodiment, the F(ab) fragment is an anti-Mouse F(ab) fragment. In another embodiment, the F(ab) fragment is an anti-Rabbit F(ab) fragment, anti-Mouse F(ab) fragment or another species specific F(ab) fragment.

In one embodiment, the F(ab) fragment is an anti-biotin F(ab) fragment.

In alternative embodiments, the fluorescent protein is selected from the group consisting of a Green Fluorescent Protein (GFP), Yellow Fluorescent Protein (YFP), Cyan Fluorescent Protein (CFP), and mCherry. In another embodiment, the fluorescent protein is a green fluorescent protein (GFP) or a similar protein. In a further embodiment, the fluorescent protein is a Green Fluorescent Protein (GFP). In an additional embodiment, the fluorescent protein is a fluorescent phycobiliprotein. In another embodiment, the fluorescent phycobiliprotein is allophycocyanin, phycocyanin, phycoerythrin, or phycoerythrocyanin.

In alternative embodiments, the fluorescent protein comprises a small fluorescent group surrounded by an inert shell that prevents the fluorophore from coming in contact with its surroundings. In another embodiment, the fluorescent protein comprises a small fluorescent group surrounded by proteins. In a further embodiment, the fluorescent protein comprises a small fluorescent group surrounded by a protein beta barrel. In further embodiments, the fluorescent label does not form a non-cleavable bond with its surroundings upon excitation with light.

In alternative embodiments, the label comprises a fluorophore surrounded by an inert shell that prevents the fluorophore from coming in contact with its surroundings. In another embodiment, the label comprises a fluorophore surrounded by proteins. In a further embodiment, the label comprises a fluorophore surrounded by a protein beta barrel. In further embodiments, the fluorophore does not form a non-cleavable bond with its surroundings upon excitation with light. In an embodiment, the fluorophore is a fluorescent group.

In another embodiment, the label is not a small-molecule fluorophore. In some embodiments, the label is a small-molecule fluorophore

The subject invention also provides a process of immunostaining a sample comprising staining the sample with a selectively cleavable probe.

The subject invention further provides a process of immunostaining a sample comprising
a) obtaining a probe comprising a primary antibody against a preselected target linked to biotin,
b) staining the sample with the probe of step a),
c) staining the sample with a selectively cleavable probe, wherein the F(ab) fragment is an anti-biotin F(ab) fragment.

In an embodiment, the primary antibody against the preselected target is conjugated to biotin or linked to biotin by a disulfide bond. In another embodiment, the primary antibody against the preselected target is linked to biotin by a disulfide bond.

In alternative embodiments, the process further comprises washing off any unbound primary antibody after step b).

In one embodiment, the process further comprises imaging the sample after staining the sample with the selectively cleavable probe.

In another embodiment, the process further comprises destaining the sample by cleaving the bond between the F(ab) fragment and the one or more fluorescent proteins.

In one embodiment the destaining comprises contacting the sample with a reducing agent. In an embodiment, the amount of the reducing agent is 5-50 mM or 20-50 mM. In another embodiment, the reducing agent is a disulfide reducing agent. In a further embodiment, the reducing agent is tris(2-carboxyethyl)phosphine (TCEP) or Dithiothreitol (DTT).

In another embodiment, the destaining is performed under near physiological conditions or under mild conditions.

In alternative embodiments, the process further comprises cleaving unbound biotin moieties on the primary antibody.

In one embodiment, the process further comprises the step of staining the sample with a permanent label that is unaffected by disulfide reducing agents. In an embodiment, the permanent label is phalloidin-Alexa 647.

In one embodiment, the permanent stain is not affected by the destaining.

In an embodiment, the target is selected from the group consisting of a protein, and an antigen.

In alternative embodiments, the process further comprises repeating the process for a second preselected target.

In alternative embodiments, the process further comprises repeating the process for a third preselected target. In another embodiment, the process further comprises repeating the process for a fourth preselected target. In a further embodiment, the process further comprises repeating the process for a fifth preselected target. In another embodiment, the process further comprises repeating the process 6 or more times for additional preselected targets.

In another embodiment, the sample is not degraded during the process.

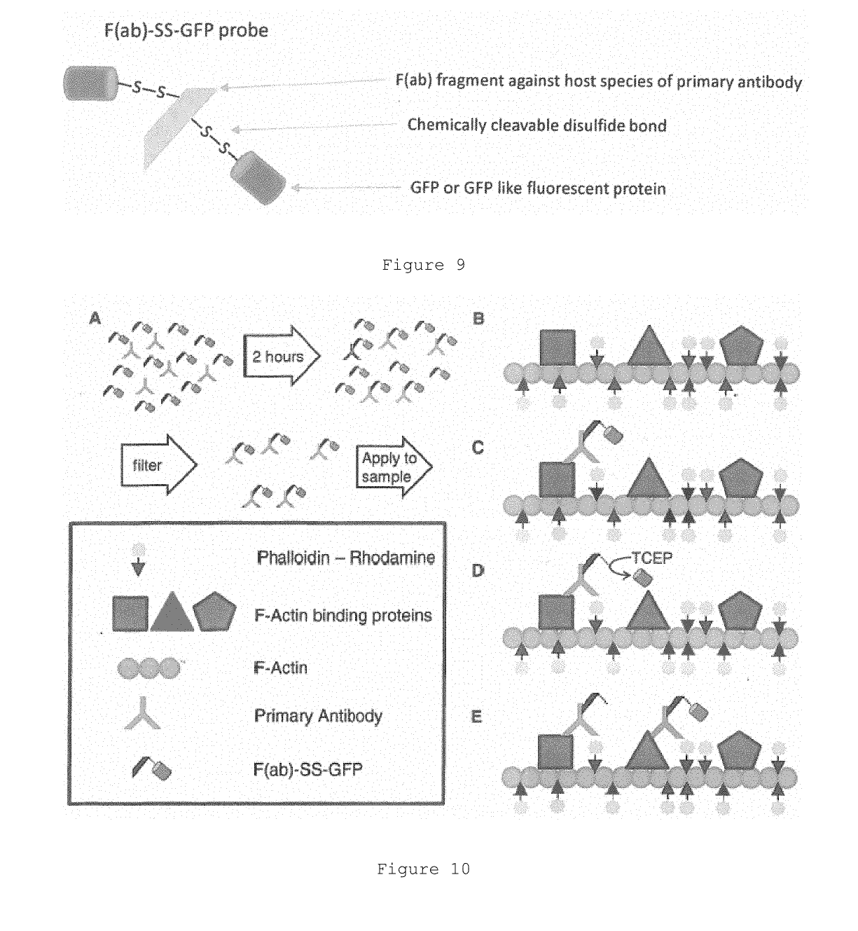

The invention further provides a selectively cleavable probe comprising an F(ab) fragment linked to one or more fluorescent proteins by a chemically cleavable disulfide bond to form a F(ab)-SS-GFP probe. See FIG. 9 for an illustration of this. The F(ab)-SS-GFP probe can be bound, in solution, to a primary antibody against a specific target in the sample. See FIG. 10 for an illustration of this.

In an embodiment, the label is a dye. In another embodiment, the label is a small molecule fluorophore. In a further embodiment, the small molecule fluorophore is fluorescein, or Cy5. The small molecule fluorophore may be any known small molecule fluorophore. In another embodiment, the F(ab) fragment is from a secondary IgG antibody. In an additional embodiment, the F(ab) fragment is a monovalent F(ab) fragment.

Any known disulfide reducing agent may be used in this the process of this invention. Many of disulfide reducing agents are commercially available. Common disulfide reducing agents include Tris(2-carboxyethyl) phosphine (TCEP), and Dithiothreitol (DTT).

Fluorescent phycobiliproteins may be used as labels. This family consists of a small fluorescent group surrounded by a large protein. Structurally, they are similar to GFP (or YFP, CFP, mCherry, etc) in that they have a small fluorescent group surrounded by proteins. Because fluorescent phycobiliproteins have this basic structural commonality where the fluorescent group is embedded inside the protein, excitation of the fluorescent group does not lead to formation of a bond with the intracellular environment.

Where a range is given in the specification it is understood that the range includes all integers and 0.1 units within that range, and any sub-range thereof. For example, a range of 77 to 90% includes 77.0%, 77.1%, 77.2%, 77.3%, 77.4%, 77.5%, 77.6%, 77.7%, 77.8%, 77.9%, 80.0%, 80.1%, 80.2%, 80.3%, 80.4%, 80.5%, 80.6%, 80.7%, 80.8%, 80.9%, and 90.0%, as well as the range 80% to 81.5% etc.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Experimental Details

### Multiplex Imaging

In sequential or iterative multiplex imaging, the typical strategy is to i) apply a single fluorescent probe to a sample, ii) image its distribution in a specific region of the sample with a fluorescence microscope, and iii) permanently deactivate the fluorescence of that probe optically or chemically (known as destaining or deactivation). This staining, imaging, destaining protocol is repeated for many different probes and ultimately generates a composite image of the distribution of many different stains.

The current generation of techniques and probes for iterative staining have several limitations:

i) Moderate to no fluorescence destaining specificity. It is often desirable to stain a sample with two types of probes—the deactivatable probes mentioned above that iteratively stain many different molecules, and one or more permanent stain(s) that will persist throughout the iterative staining process e.g. to label a specific cell type or organelle. Current approaches for iterative staining do not permit the selective and complete destaining of the iterative probe without affecting the permanent probe.

ii) The iterative stain cannot be completely deactivated under mild conditions. Typically, in order to completely destain the sample, severe buffer conditions are required (low pH, high osmolarity, etc.) (Micheva & Smith, 2007) that can damage the sample itself. Moreover, as shown below, the very act of imaging a fluorophore creates a reactive excited state of that fluorophore that can cause it to covalently bind to other nearby intracellular molecules (Holden & Cremer, 2003). This covalent binding of the fluorophore to its surroundings makes it very difficult to chemically remove. Hence, the very act of imaging a destainable fluorophore can make it non-destainable.

To overcome these limitations, we have developed an iterative staining approach, and a class of fluorescent probes that can be completely and selectively deactivated on a sample under mild buffer conditions. These probes can be used for sequential multiplex imaging and offer the advantage that the sample is not exposed to harsh treatments, and other conventional, permanent stains on the sample will not be affected.

### Example 1

The gold standard for imaging protein-protein interactions in cells is Förster Resonance Energy Transfer (FRET), which is sensitive to distances of up to ˜100 Å between putatively interacting molecules. Because FRET requires two interacting molecules to be labeled with two different colors, the number of distinct binary interactions that can be imaged simultaneously on the same sample is limited by the number of colors that can be spectrally separately resolved within the wavelength range detectable by conventional fluorescence detectors. Currently, only a maximum of two separate FRET pairs have been convincingly, simultaneously imaged on the same sample (Grant 2008). Simultaneous imaging of a larger number of putative interactions would require a correspondingly larger palate of label colors.

A superior alternative to simultaneous labeling is sequential labeling in which a single sample is iteratively stained, imaged and then de-stained. It is relatively straightforward to image the same field of view in the sample and spatially co-align the images acquired from each imaging round during post-processing. Using this sequential staining approach, many stains (>10) can be imaged on a given sample, yielding a final composite spatial map of a large number of different proteins. In these techniques, de-staining is accomplished either by antibody elution buffers (Micheva 2010, Wahlby 2002) which strip the antibody probes from the sample or chemical deactivation of the fluorophore (Gerdes 2013) which destroys the fluorescent label on the antibody probe.

To measure several interactions with a given target protein, it is necessary to be able to sequentially stain and de-stain the interactors, without affecting the label on the target. The existing approaches for sequential staining therefore cannot be used to measure multiple binary interactions as their de-staining methods non-selectively destroy all stains on the sample. Elution buffers use low pH or high salt concentrations to partially denature proteins in the sample and thereby undo antibody-antigen binding. These harsh, denaturing conditions will also de-stain the target, and over repeated rounds will physically degrade the sample. Chemical deactivation of fluorophores is generally milder. In fact Gerdes et al (Gerdes 2013) have demonstrated that it is possible to sequentially stain a sample for 61 antigens. However, as before, while different fluorescent probes are differently susceptible to different chemical deactivation buffers, it is rare to find robust FRET pairs in which one fluorescent label can be entirely deactivated by a buffer while the other is unaffected by the same buffer.

The currently available methods discussed above cannot image more than two protein-protein interactions on the same sample using FRET. Simultaneous staining approaches lack an adequate spectral range, and sequential staining approaches lack sufficiently selective de-staining buffers. The sequential target interaction imaging method that we have begun to develop overcomes several of these limitations. The approach in the following Examples allows the interactors to be selectively de-stained with high specificity, causing no damage to either the target stain or any other ancillary stains on the sample (e.g. synaptic markers or whole cell labels). Moreover, a wide range of fluorophores can be used, allowing FRET pairs to be chosen at whatever wavelength is most convenient for the user. The techniques in the follow examples enables, for the first time, the imaging of many protein-protein interactions on the same region of interest and will thereby enable the reconstruction of the interactomes of key proteins important for neuronal function and identity.

### Example 2

Immunostains are conventionally done as, first, a stain with a primary antibody against a particular antigen, followed by another stain with a fluorescent secondary antibody against the host species of the primary antibody. An alternative to this staining protocol, that presents key advantages, involves using fluorescently labeled monovalent Fab fragments instead of conventional IgG secondary antibodies (Brown 2004). Monovalent Fab fragments are the ˜50 kD fragments of IgG antibodies that contain a single antigen binding site per fragment. They are obtained by digesting IgG antibodies with papain to sever the two Fab fragments from the Fc fragment Staining a specific antigen using Fab fragments (FIG. 10A) requires first mixing in solution a primary antibody against that antigen with a fluorescently labeled Fab fragment against the primary host species. Because Fab fragments are monovalent (have a single antigen binding site) they bind to the primary antibodies without inducing aggregation of the antibody. Once the primary antibody and labeled Fab form a bound complex, the unbound Fab can be removed by ultrafiltration through a molecular weight filter with a 100 kD cut off. The filtered primary antibody+Fab complex can then be applied directly to the sample to stain the specific antigen.

The procedure of this example follows this general staining protocol, except we use monovalent Fab fragments that have been labeled with Fluorescein via a cleavable disulfide bond using standard disulfide labeling chemistry. Monovalent Fab fragments naturally contain a single disulfide bridge between proximate cysteine residues that is accessible under non-denaturing conditions (Liu 2012). Disulfide reducing agents such as TCEP or DTT reduce this superficial disulfide bond (-S-S-) to thiols (-SH), which are then reacted with fluorescein methanethiosulfonate to produce Fab fragments labeled with Fluorescein via disulfide bonds (Fab-SS-Fluorescein). While Fab-SS-Fluorescein is a stable conjugate under normal conditions, Fluorescein can be cleaved from the Fab fragment using the same disulfide reducing agents (TCEP or DTT) used to cleave the original disulfide bridge on the Fab fragment.

As an illustration, FIGS. 10B-E outline the sequential imaging of interactions between a target, f-Actin, and multiple f-Actin binding proteins. The target, f-Actin is first stained with a conventional fluorescent probe such as Phalloidin-CF568 (Ex/Em=562/583) (FIG. 10B). Then a primary antibody+Fab-SS-Fluorescein complex is generated as described above and used to stain the first f-Actin binding protein (FIG. 10C). The interaction between f-Actin and the first f-Actin binding protein can now be measured using FRET or co-localization, after which the Fluorescein label on the Fab fragment is selectively cleaved using TCEP (FIG. 10D) leaving the f-Actin stain intact. The sample can then be stained with a primary antibody+Fab-SS-Fluorescein complex against the second f-Actin binding protein. This process can be repeated for many interactors.

The crucial advantage of using a Fab based staining protocol over a traditional primary+secondary antibody staining protocol now becomes clear. The complex of the primary antibody and fluorescent Fab is ultra-filtered to remove any unbound Fab prior to applying the complex to the sample. Hence there is no free fluorescent Fab in the staining solution applied to the sample that might stain primary antibodies deposited during previous staining rounds. This strategy ensures that only a single interactor is stained on any given round, even if the primary antibodies used in previous staining rounds derived from the same host species.

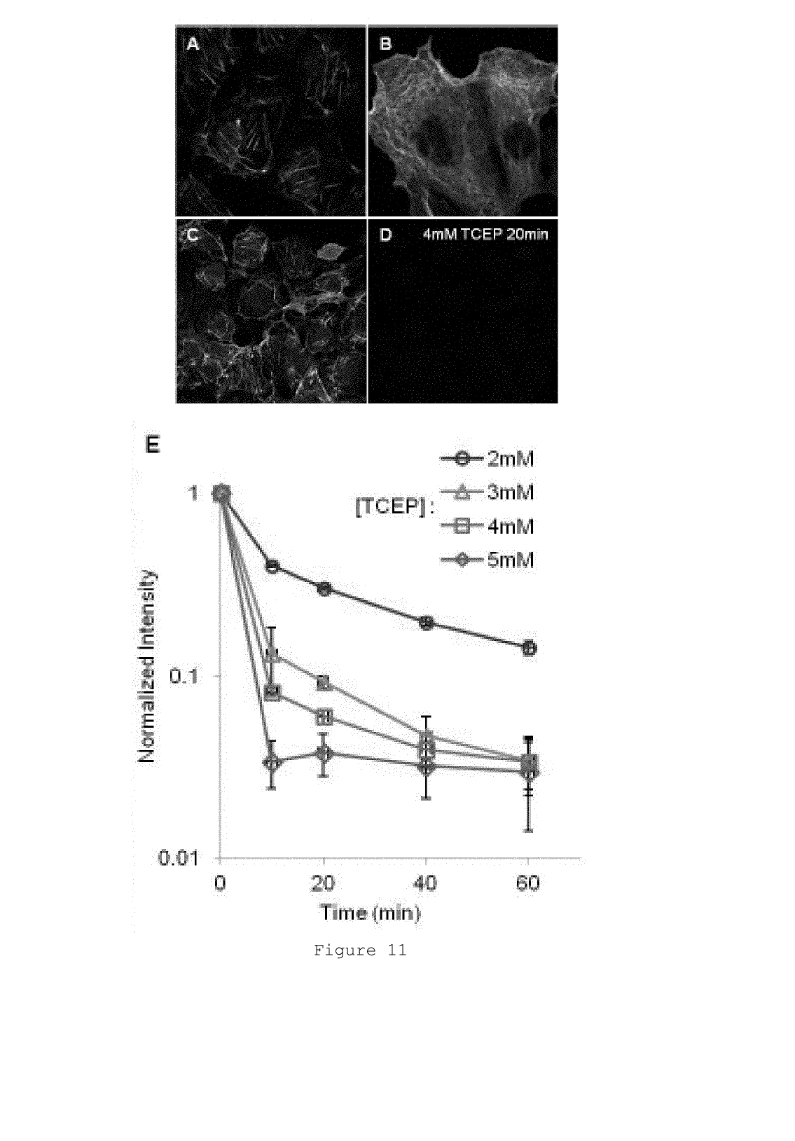

The covalent modification of a Fab fragment potentially alters its affinity for its antigen. Since only one superficial disulfide bridge per Fab fragment is modified, its structure and function are not likely to be affected. To confirm the functionality of Fab-SS-Fluorescein conjugates, COS-7 cells were stained with anti-Myosin 2b (FIG. 11A) and anti-Tubulin (FIG. 11B) primary antibodies. The cells were then stained with Fab-SS-Fluorescein (green) and conventional Cy3 labeled IgG secondary antibodies (red), both against the primary antibody host species. The high degree of co-localization (yellow) between the Fab-SS-Fluorescein and IgG-Cy3 images indicates the Fab fragments stain their target with as much fidelity as conventional secondary antibodies.

Fab-SS-Fluorescein stained samples can be de-stained using the disulfide reducing agent TCEP. A stain against Myosin 2b in COS-7 cells using Fab-SS-Fluorescein (FIG. 11C) can be almost completely de-stained in 20 minutes using even a low concentration of TCEP (FIG. 11D). The amount of de-staining depends on time and the concentration of TCEP (FIG. 11E), however we find that even modest concentrations of TCEP (5 mM) can lead to almost complete (˜97%) de-staining within 20 minutes. Furthermore, this disulfide reducing agent (TCEP) is highly specific and has no effect on other conventional fluorescent labels that do not contain disulfide bonds (most do not). Labeling with Fab-SS-Fluorescein is therefore potentially a gentle, selective method for iteratively staining and de-staining a sample.

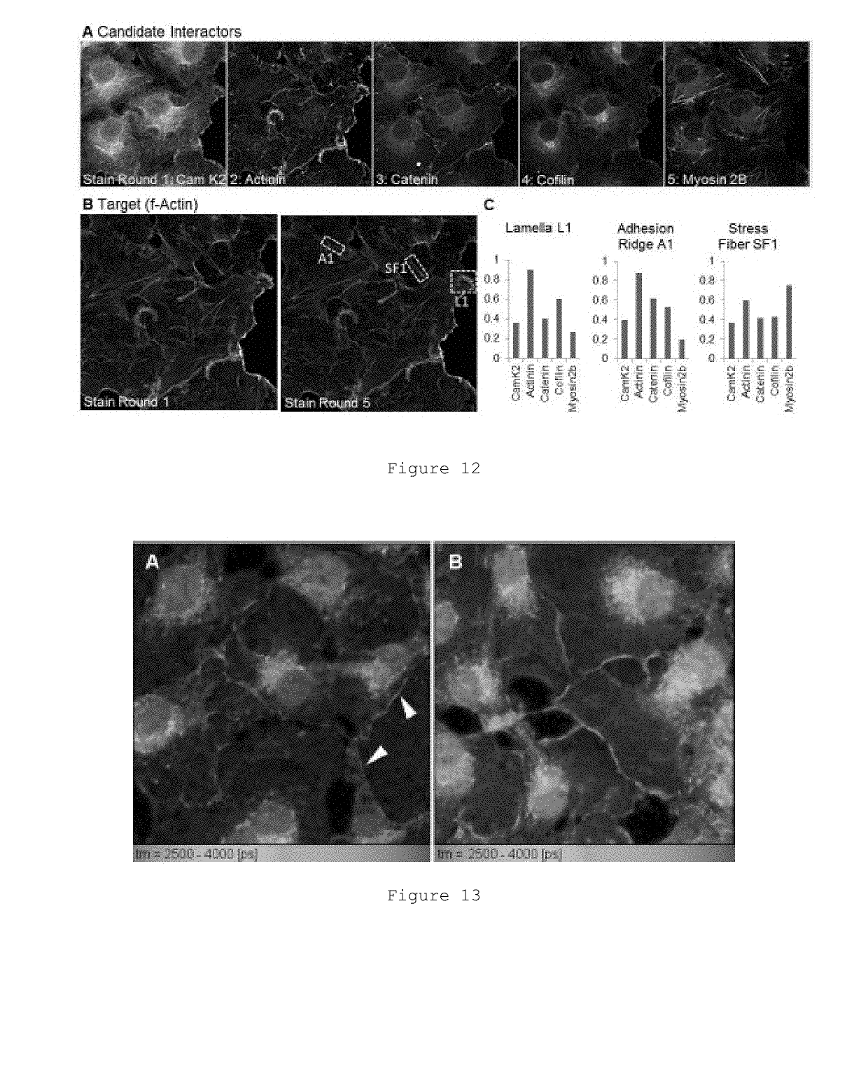

To demonstrate a set of sequential interaction measurements in which interactors are iteratively stained and de-stained while the target is unaffected, f-Actin was chosen as the target, since it is one of the most promiscuous interactors in the cell and has a demonstrable role in numerous forms of neuronal plasticity (Ramachandran 2009, Fischer 1998, Fischer 2000, Okamoto 2004, Lin 2010). F-Actin was first stained with the small molecule Phalloidin-Alexa 647. The first candidate interactor was then chosen, in this case Cam Kinase 2 (β/γ/δ pan antibody) (FIG. 12A, Stain Round 1: Cam Kinase 2), and stained using the anti-Cam Kinase 2+Fab-SS-Fluorescein complex formed using the reaction protocol outlined in FIG. 10A. Images of the target and interactor were acquired, and then the fluorescent label on the Fab was cleaved using TCEP, thereby de-staining the Cam Kinase 2. The second candidate interactor was then chosen: α-Actinin, stained, imaged, and de-stained. This process can be repeated as often as required—in this case we stained and imaged the same field of view for CamK2, α-Actinin, α-N-Catenin, Cofilin and Myosin 2B. On each round (FIG. 12A, Stain Round 1-5), we obtained the spatial distribution of another candidate interactor and then eliminated the interactor stain while leaving the target staining intact. FIG. 12B. shows that after five rounds of interactor staining and de-staining, the Phalloidin-Alexa 647 target stain was minimally affected, showing only a ˜15% decrease in intensity.

During the initial development of this method, the interaction between f-Actin and any given interactor in a region of interest was defined as the co-localization (Pearson's correlation coefficient) of the two fluorescence signals in that region of interest. While co-localization is a poor measure of protein-protein interaction, we use it as a preliminary, qualitative indicator of interaction and plan to modify our method to use FRET to quantitatively measure interactions. FIG. 12C shows the interaction (co-localization) of f-Actin with a number of other proteins in several regions of the cell. In a lamella, a particularly strong interaction of f-Actin with α-Actinin and a moderate interaction with Cofilin was found. In an adhesion ridge between two cells a high interaction with α-Actinin and a moderate interaction with α-N-Catenin and Cofilin was found. And on a stress fiber, a high interaction with Myosin 2b and a moderate interaction with α-Actinin was found. Cam Kinase 2 does not show high co-localization with f-Actin in any of these regions, indicating only weak or no interaction of the β/γ/δ isoforms with f-Actin in COS-7 cells. Even with this relatively small number of interactors, we see that the f-Actin interactome varies between distinct functional structures within the cell (FIG. 12C).

While co-localization of two fluorophores in a confocal microscope image is necessary for interaction, it is not generally a good measure of interactions because the spatial resolution of a confocal fluorescence microscope (˜200 nm) is much greater than the typical distance between interacting proteins (<100 Å). Two fluorophores may appear to co-localize even though they may be up to ˜200 nm apart, giving a false positive indication of interaction. We therefore plan to use Förster Resonance Energy Transfer (FRET), sensitive to distances less than 100 Å, to measure protein-protein interactions.

f-Actin was stained with a FRET acceptor label (Phalloidin-CF568) and α-N-Catenin with a primary antibody+Fab-SS-Fluorescein complex that serves as the FRET donor to demonstrate the feasibility of using FRET to detect the interaction of a target with a Fab-SS-Fluorescein stained interactor (FIG. 13). Using fluorescence lifetime imaging (FLIM) of the donor fluorescence (Sun 2011, Lakowicz 1999), FRET at a given pixel can be detected as a decrease in the donor fluorescence lifetime at that pixel. The fluorescence lifetime image of cells stained only for α-N-Catenin with the antibody+Fab-SS-Fluorescein complex (FIG. 13A) displays an average lifetime (tm) of ˜3.5 ns along the adhesion ridges between cells (white arrows). When f-Actin is also stained with Phalloidin-CF568 on the same sample, FRET from the α-N-Catenin donor label to the f-Actin acceptor label reduces the average lifetime along these adhesion ridges to ˜3.1-3.3 ns (FIG. 13B, red arrows).

Using FLIM, it will also be possible to estimate the population fraction of the interactor (donor) that is bound to the target (acceptor) at every pixel. While the use of FLIM to quantitatively study protein-protein interaction is well established, combining sequential Fab staining with the FLIM imaging of FRET poses several optimization challenges largely related to fluorophore brightness and stability.

### Example 3

### Imaging Molecular Interaction Networks with Serial Target Interaction Microscopy (STIM)

Procedure:

1) Label the Target with any conventional fluorescent probe;

2) Label Interactor 1 with STIM antibody against Interactor 1. Measure interaction strength between Target and Interactor 1;

3) Cleave fluorescent label on STIM antibody;

4) Label Interactor 2 with STIM antibody against Interactor 2. Measure interaction strength between Target and Interactor 2;

5) Cleave fluorescent label on STIM antibody;

6) Label Interactor 3 with STIM antibody against Interactor 3. Measure interaction strength between Target and Interactor 3.

7) Repeat process.

Antibodies for STIM may be a conventional antibody raised in mouse/rabbit and anti mouse/rabbit Fab fragment labeled with dye via cleavable disulfide bond. The antibodies are then incubated for 1-3 hours at room temperature. Next, unbound F(ab) fragments are filtered out and the remaining antibodies for STIM are applied to the cell sample. STIM probes were found to be as robust as conventional antibody probes and they were cleavable by disulfide reducers. The sample was able to be re-probed.

### Example 4

### Probe Design and Staining Protocol

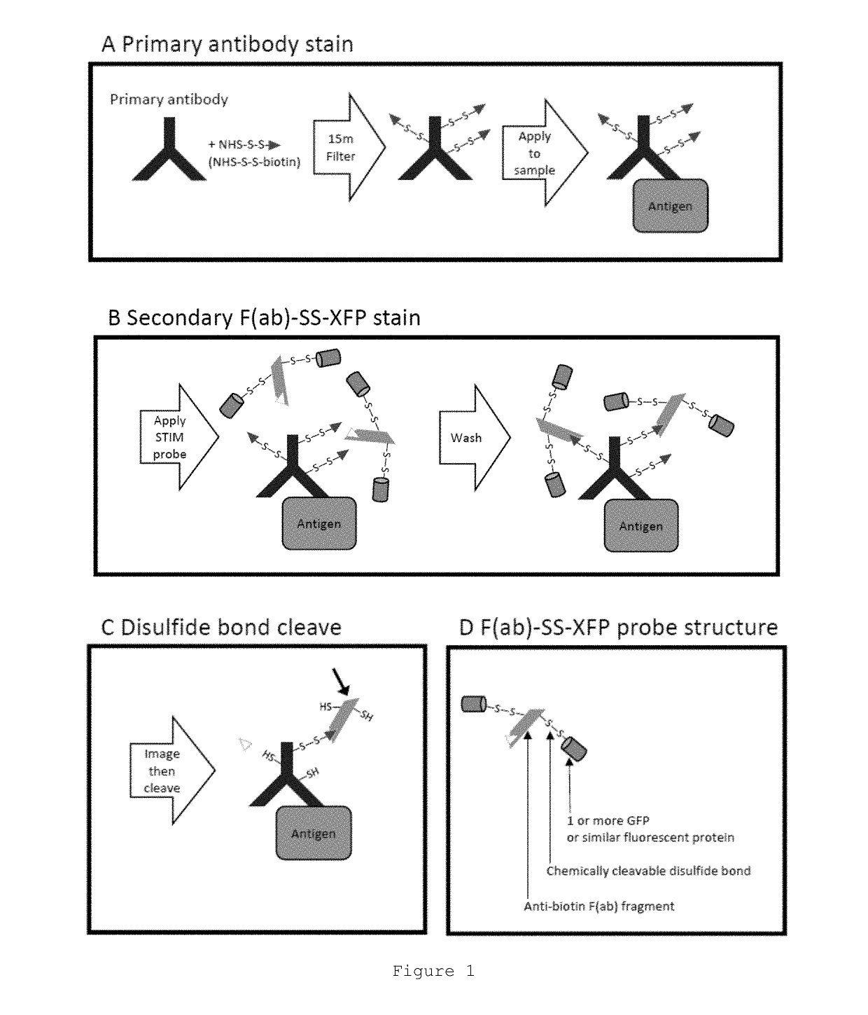

The fluorescent probe developed (FIG. 1D) consists of an anti-biotin monovalent F(ab) fragment from an IgG antibody that is linked to one or more GFPs (or a similar fluorescent protein: ‘XFP’) via disulfide bonds yielding an anti-biotin F(ab)-SS-GFP probe. An immunostain is then accomplished in two staining rounds. In the first round (FIG. 1A), a primary antibody against a chosen antigen in the sample is conjugated to biotin via cleavable disulfide bonds. The resulting cleavably biotinylated primary antibody is then used to stain the sample as would be done in a conventional immunostain. After the primary incubation, any unbound primary antibody is washed off, and the anti-biotin F(ab)-SS-GFP probe is applied (FIG. 1B). This secondary incubation leaves the antigen fluorescently labeled, and ready to be imaged.

After the sample is imaged, it can be destained (FIG. 1C) by applying a disulfide reducing agent (e.g. TCEP, DTT) under near physiological conditions to cleave the bond between the F(ab) and the GFP, allowing GFP to be washed away and leaving the sample dark. Unbound biotin moeities on the primary antibody are also cleaved, preventing their binding of anti-biotin F(ab)-SS-GFP in successive staining rounds. The binding of a biotin binding protein (e.g. anti-biotin F(ab) or Streptavidin) can sterically inhibit the cleaving of the disulfide bond between the primary antibody and biotin (data not shown). In this case, some residual F(ab) can remain bound to the primary antibody (FIG. 1C, black arrow). However this residual F(ab) has been stripped of GFP and is therefore no longer fluorescent and so it does not interfere with subsequent imaging. The use of a fluorescent protein such as GFP as the fluorophore instead of a small molecule fluorophore (fluorescein, Cy5, etc.) allows complete destaining of the sample that cannot be accomplished with small-molecule fluorophores (discussed below).

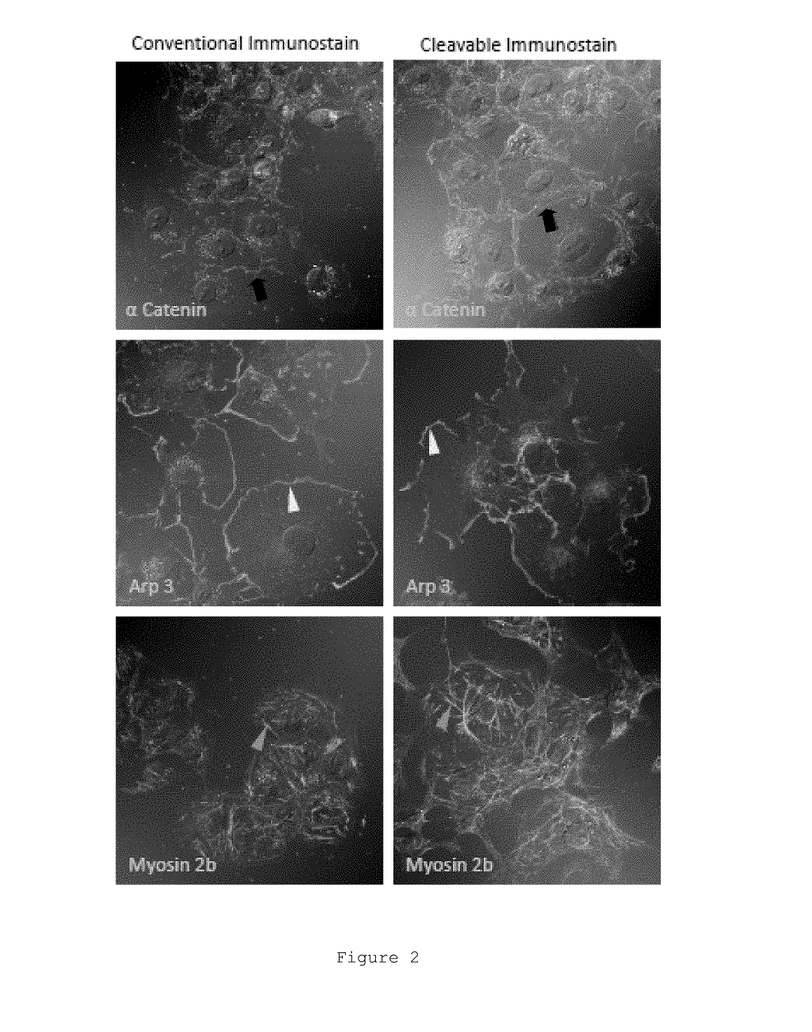

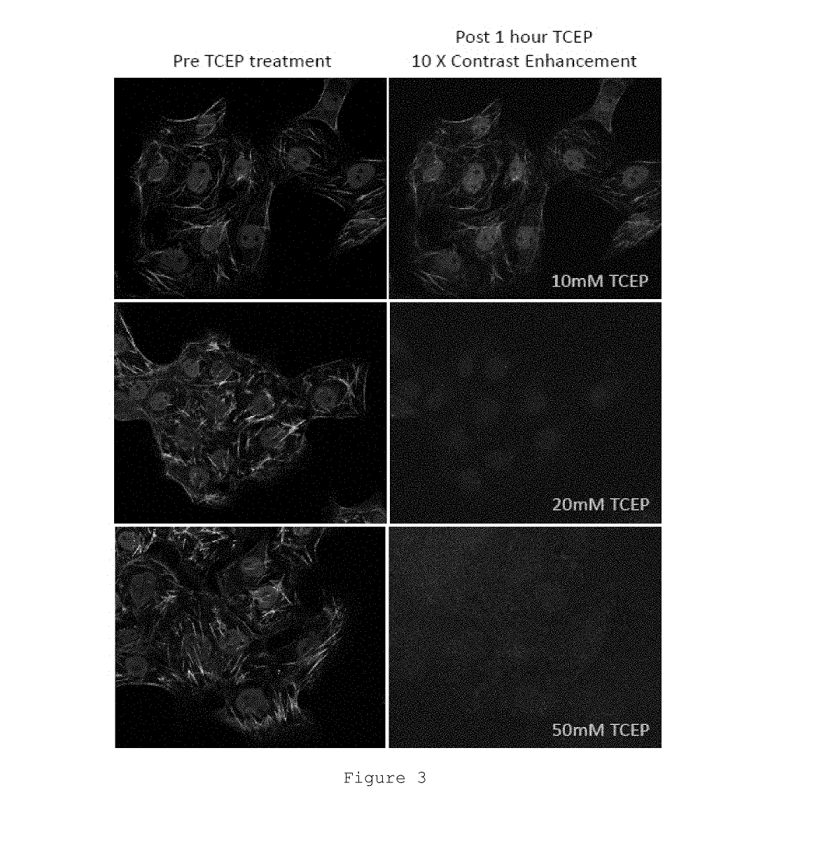

Immunostains done using a biotinylated primary and an anti-biotin F(ab)-SS-GFP exhibit the same robust specificity as immunostains done using conventional primary and secondary antibodies. By way of example, using either staining approach, stains for a Catenin, Arp 3, and Myosin 2b in COS-7 cells (FIG. 2) exhibit the characteristic intracellular distributions of these three proteins. The α N-Catenin stains shows characteristic adhesion ridges between adjacent cells. The Arp 3 stain shows enrichment in the growing edges of cells. The Myosin 2b stain shows localization to stress fibers. These stains demonstrate that biotinylation of the primary antibody and GFP conjugation to a F(ab) fragment, at the appropriate levels, can generate the same specificity as conventional antibody probes.

The feature of the anti-biotin F(ab)-SS-XFP staining system most relevant to serial staining is the ability to destain the sample under mild conditions. Destaining is effected by cleaving the GFP off the sample using a disulfide reducing agent such as TCEP or DTT. The effectiveness of this cleaving depends on both time and concentration of the disulfide reducing agent. It was found that a 1 hour wash with 20-50 mM TCEP (in 100 mM Tris-HCl, 150 mM NaCl, pH 7.4, 0.3% Triton-X 100) is adequate to remove essentially all of the GFP signal. A similar quality of staining and destaining occurs with anti-biotin F(ab)-SS-mCherry (FIG. 7), although mCherry leaves a slightly higher non-specific background staining that is visible upon contrast enhancement.

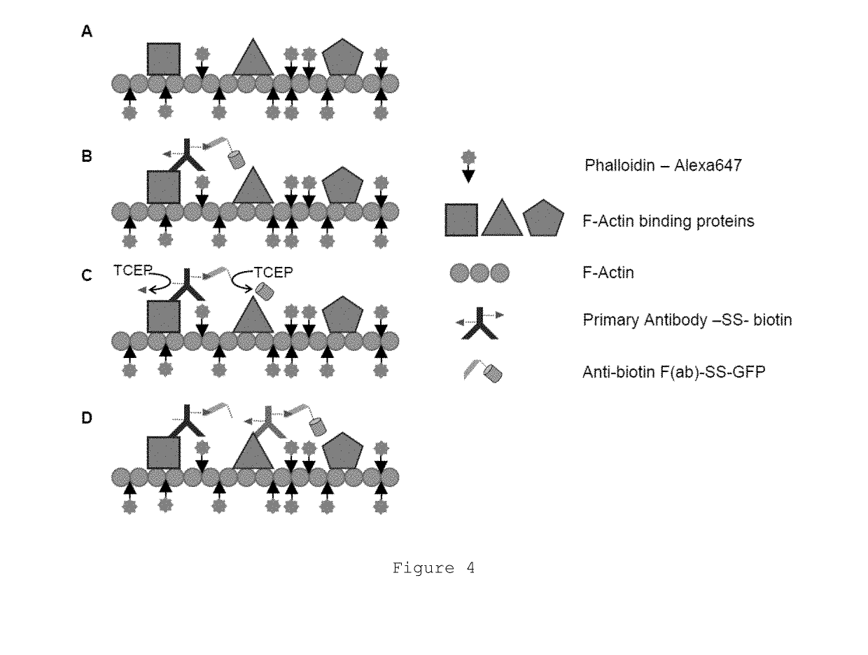

An iterative serial stain involves repeatedly staining, imaging, and destaining the same region of a sample with multiple cleavable stains (FIG. 4). The sample is first stained with whatever permanent labels are required (FIG. 4A). These permanent labels can be any conventional fluorescence probes that are unaffected by disulfide reducing agents (most are not), regardless of whether they are derived from antibodies, oligos, or small molecule probes. Since this permanent stain is unaffected by destaining, it can be used as a map to locate the same field of view in each imaging round. In the example depicted in FIG. 4, f-actin is stained permanently using Phalloidin-Alexa 647. The sample is then probed for the first protein of interest using an appropriate cleavably biotinylated primary antibody and an anti-biotin F(ab)-SS-GFP (FIG. 4B). The sample is then imaged with a fluorescence microscope to record the spatial distribution of the first protein of interest and the permanent stain. The sample is then destained with TCEP (FIG. 4C) leaving only the permanent stain, and then restained for the next protein of interest (FIG. 4D).

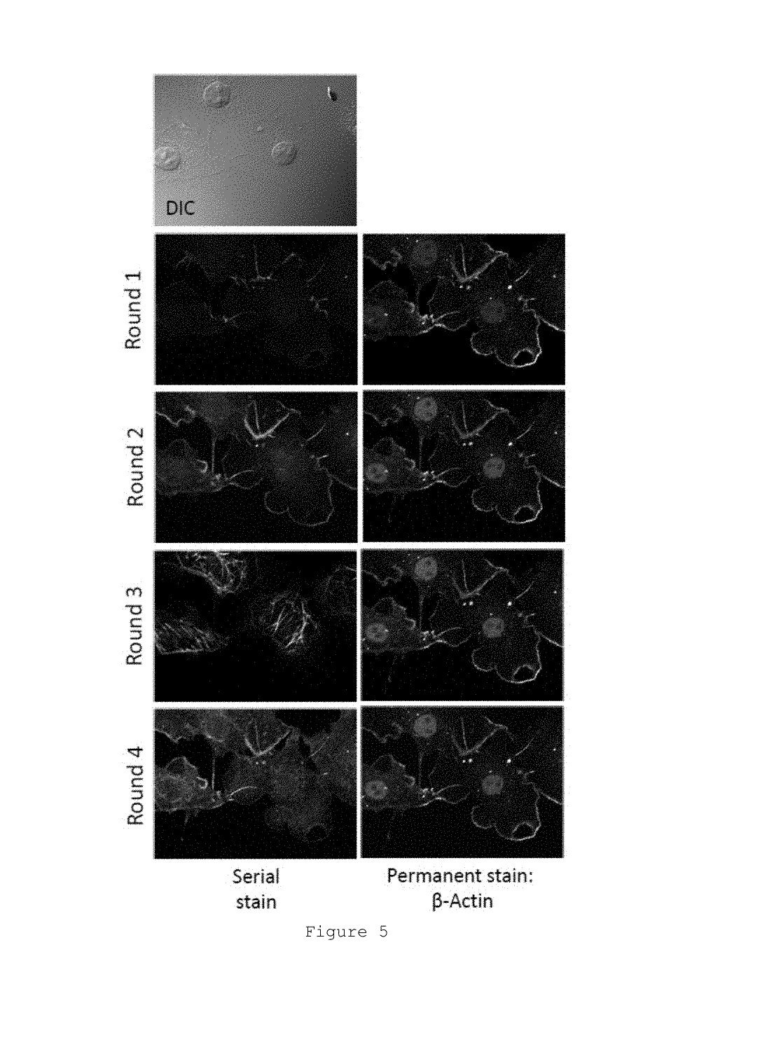

To illustrate this process (FIG. 5), fixed COS-7 cells were stained with a permanent stain against β-Actin using a conventional primary antibody (Cell Signaling Technology #3700) and a species specific secondary antibody labeled with a conventional fluorophore (Alexa 647; Invitrogen A31571). The sample was then iteratively stained, imaged, and destained to record the distributions of a Catenin, Arp 3, Myosin 2b, and Drebrin A/E. The permanent stain (right column) shows little change from the first imaging round to the last, with the exception of some minor photobleaching. The cleavable stains (left column) clearly show the typical intracellular distributions of these four proteins with virtually no carry over of signal from one round to the next. For example, the Myosin 2b stress fibers imaged in Round 3 are an order of magnitude brighter than the Drebrin A/E stain imaged in Round 4, and yet there is no residual staining of stress fibers in the Drebrin A/E image, indicating the Myosin 2b stain was completely destained.

An automated stage was used to return to approximately the same field of view in each imaging round. Fine offsets in the field of view from round to round can be compensated for by software using the permanent stain as a landmark map.

### Fluorescent Proteins are a Superior Alternative to Small-Molecule Fluorophores for Cleavable F(ab) Labeling

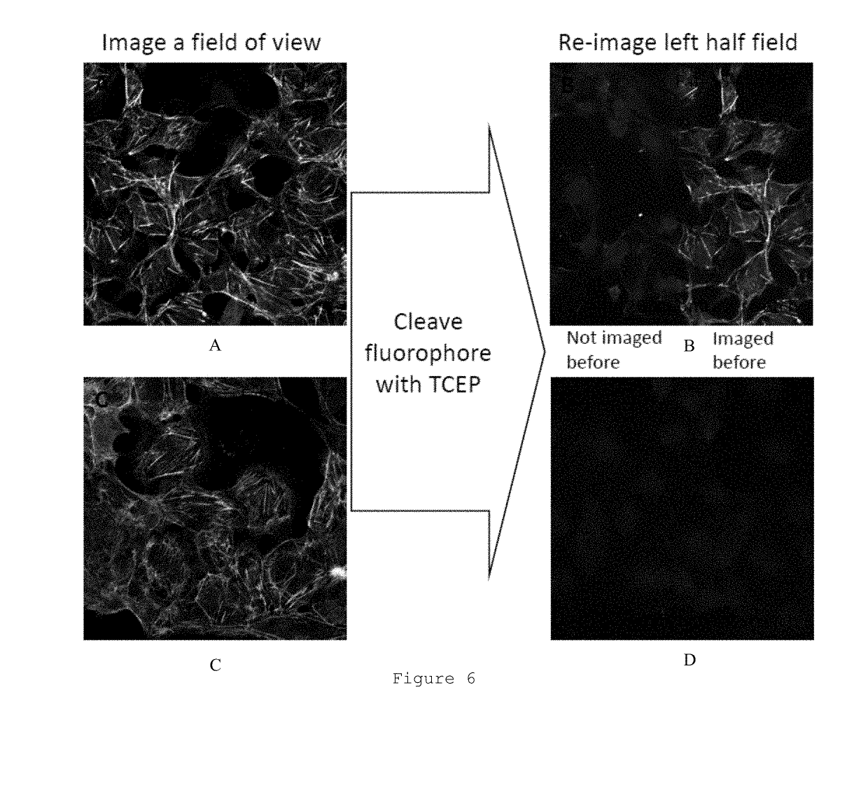

A critical feature of the probes we have developed is the use of a fluorescent protein (e.g. GFP) instead of a conventional small-molecule fluorophore (e.g. fluorescein, Cy5) to label the F(ab) fragments. Small-molecule fluorophores were found to become partially non-cleavable when exposed to excitation light during imaging. As an illustrative comparison, CF488A (a green fluorophore similar to fluorescein) and GFP are used to label anti-Rabbit F(ab) fragments, generating anti-Rabbit F(ab)-SS-CF488A and F(ab)-SS-GFP. These F(ab) fragments are then used to stain a Rabbit primary antibody against Myosin 2b in fixed COS-7 cells (FIGS. 6A and 6C). Both fluorescent F(ab) probes give comparable stains typical of the known Myosin 2b distribution in COS-7 cells. After imaging a field of view in the sample, the fluorophore on the entire sample is removed by bath applying the disulfide cleaving agent TCEP. This should in principle leave the entire sample dark as all the fluorescent molecules have been cleaved off the F(ab) fragments and washed away. However when we return to the same fields of view that were imaged prior to TCEP treatment, we find that a residual signal remains in the sample labeled with F(ab)-SS-CF488A (FIG. 6B), while the sample stained with F(ab)-SS-GFP is entirely dark (FIG. 6D). Moreover if we shift the field of view to the left by a half-frame (FIG. 6B), we find that the cells that had not been imaged prior to TCEP treatment (left half of field in FIG. 6B) are completely dark, whereas those cells that had been imaged prior to TCEP treatment alone have a residual Myosin 2b signal (right half of field in FIG. 6B).

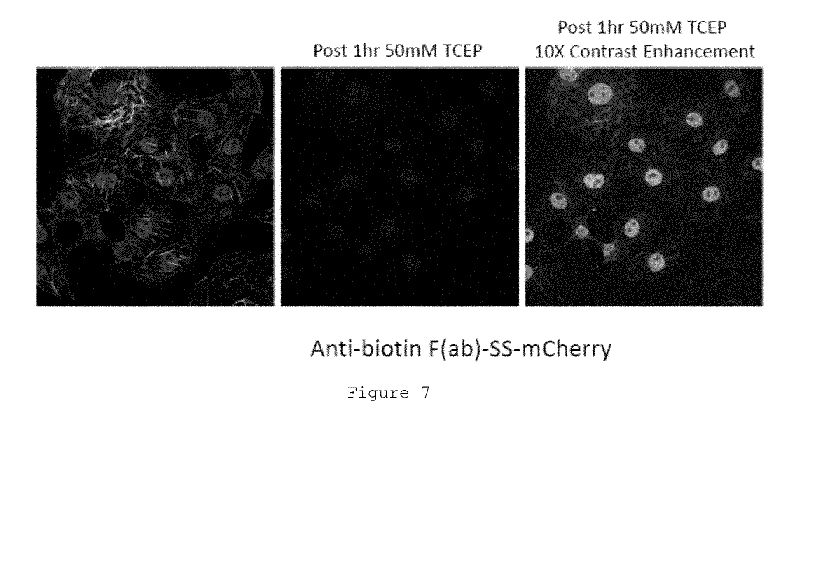

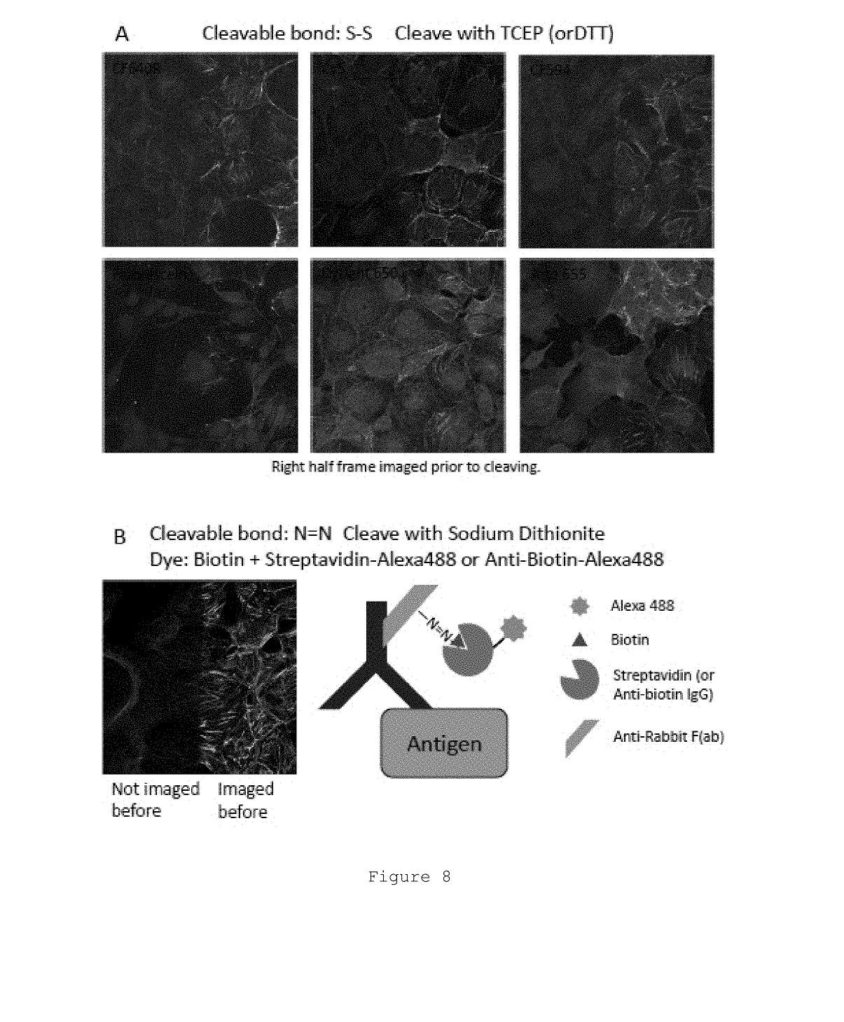

FIG. 6B indicates that the very act of imaging the cleavable F(ab)-SS-CF488A stain makes it partially non-destainable. The magnitude of the residual stain intensity is only ˜5% of the original stain intensity, hence the contrast is enhanced in FIGS. 6B and 6D to better show the residual stain. While 5% is a modest residual signal, it represents an unacceptable level of background for the next staining round. This excitation induced formation of a non-destainable stain is not unique to CF488A. In fact, every small molecule fluorophore we have tried exhibits this phenomenon to some extent (FIG. 8).

The excitation induced formation of a non-destainable fluorescence does not specifically require a disulfide bond nor even a direct covalent link between the excited fluorophore and the cleavable bond. We generated a destainable stain of Myosin 2b using an anti-Rabbit F(ab) fragment labeled with biotin via a cleavable diazo bond (FIG. 8). This biotin could then be stained with Alexa 488 labeled Streptavidin or an Anti-biotin IgG. In this case, the cleavable bond is a diazo bond and there is no covalent path between the cleavable bond and the fluorophore. Nonetheless, areas of the sample that had been imaged prior to destaining showed a significant non-destainable residual Myosin 2b stain.

While the mechanism of the excitation induced formation of a non-cleavable bond is not understood, it was hypothesized that upon excitation with light, the fluorophore enters a reactive excited state that can form a non-cleavable covalent bond with nearby intracellular molecules (proteins, etc). While the disulfide bond is cleavable by TCEP, this new bond is not, and hence the fluorophore cannot be fully removed by TCEP. This phenomenon has been observed and even utilized for the light induced patterning of a protein coated surface with a fluorophore (Holden & Cremer, 2003; Jayagopal, Stone, & Haselton, 2008).

If the small-molecule fluorophore becomes non-cleavable because its excited state reacts with its surroundings to form a covalent bond, then it follows that preventing the excited state fluorophore from coming into contact with the intracellular environment will prevent the formation of such a non-cleavable bond. Fluorescent proteins such as GFP (mCherry, YFP, CFP, etc) consist of a small fluorescent group surrounded by a protein beta barrel. This beta barrel serves as an inert shell that prevents the fluorophore from coming into contact with its intracellular surroundings. Hence GFP should not form any non-cleavable bonds with its surroundings upon excitation with light. Indeed, we find that F(ab)-SS-GFP can be fully cleaved and removed from the sample (FIG. 6D) after imaging. Fluorescent proteins such as GFP are therefore a superior alternative to small molecule fluorophores as fully and selectively cleavable labels of F(ab) probes.

This light induced formation of a non-cleavable bond in the case of small-molecule cleavable fluorophores has not been previously observed or reported in the literature, and therefore the solution to this problem is novel, inventive and non-obvious.

This technology can be used where multiplexing technologies are currently used with small molecule fluorophores, such as in screening for cancer cells, or where primary antibodies exist for the biological target of interest.

### Materials & Methods

### Cleavable Biotinylation of Primary Antibodies

Primary antibodies were purchased against Myosin 2b (Sigma-Aldrich M7939), a Catenin (Assay Biotech C0137), Drebrin A/E (Millipore AB10140), Arp 3 (Abeam ab49671). Care was taken to select only antibodies that do not have other proteins (e.g. BSA) in the storage buffer. These antibodies were cleavably biotinylated using EZ-Link Sulfo-NHS-SS-Biotin (Thermo Fisher Scientific 21331).

Small volumes of these antibodies were cleavably biotinylated to test the optimal level of biotinylation. Too little biotinylation results in a weak fluorescence signal due to insufficient anti-biotin F(ab)-SS-GFP binding. Too much biotinylation can disrupt the primary antibody binding of its antigen, also leading to a low and non-specific fluorescence. 1 μl of 1 mg/ml primary antibody was mixed with 1 μl of 1 mM, 3 mM, and 10 mM EZ-Link Sulfo-NHS-SS-Biotin in PBS pH 7.4. The mixture was incubated at room temperature for 15 minutes. 10 ul of (100 mM Tris-HCl+150 mM NaCl, pH 7.4) was then added for 5 minutes to stop the reaction and quench any unconjugated Sulfo-NHS-SS-Biotin. The entire volume was then filtered through a 40 kD Zeba Micro Spin Desalting Column (Thermo Fisher Scientific 87764) to remove any unbound Biotin and exchange the buffer to PBS pH 7.4.

Each of these biotinylated primary antibodies were used to stain samples of fixed COS-7 cells with anti-biotin F(ab)-SS-GFP (see staining protocol). The samples were then imaged to determine which level of biotinylation gave the most fluorescence signal.

### F(ab)-SS-XFP Synthesis

F(ab)-SS-XFP was synthesized by linking anti-biotin F(ab) to recombinant GFP (or mCherry) using the Solulink linker technology. 180 μM recombinant GFP (or 240 μM mCherry) was mixed with 2 mM S-HyNic (Solulink S-1002-010; from 20 mM stock in DMSO) for 3 hours at room temperature. Excess unreacted S-HyNic was removed by filtering the mixture through Micro Bio-Spin P-6 Gel Columns (Bio-Rad 7326221) twice. This generated 60 μM GFP-HN (or 90 μM mCherry-HN) as measured by absorbance.

Anti-biotin F(ab) (Rockland 800-101-098) was concentrated to 4 mg/ml using an Amicon Ultra 0.5 ml 10K centrifugal filter (Millipore UFC501024) and then mixed with 1 mM S-SS-4FB (Solulink S-1037-010; from 20 mM stock in DMF) for 3 hours at room temperature. Excess unreacted S-SS-4FB was removed by filtering the mixture through Micro Bio-Spin P-6 Gel Columns twice. This generated 30 μM anti-biotin F(ab)-SS-4FB as measured by absorbance.

Anti-biotin F(ab)-SS-GFP was then generated by mixing 5 μl anti-biotin F(ab)-SS-4FB+2 μl GFP-HN+2 ul PBS+1 ul 10× Turbolink Catalyst Buffer (Solulink 5-2006-105) for 30 minutes at room temperature. The mixture was then filtered through a 50 kD Amicon Ultra centrifugal filter (Milipore UFC505024) for 3 10-minute spins to remove any unconjugated F(ab) and GFP.

### Staining Protocol

COS-7 cells were grown on glass substrates until they were ˜50% confluent. Cells were fixed in 4% PFA in PBS for 15 minutes at room temperature. The cells were then permeablized and blocked with Blockaid (Thermo Fisher Scientific B-10710)+0.3% Triton-X 100 for 1 hour at room temperature. Staining of the cleavably biotinylated primary was done overnight at 4° C. or for 3 hours at room temperature at 1/100- 1/300 dilution into Blockaid+0.3% Triton-X 100. After a thorough wash in PBS, the anti-biotin F(ab)-SS-GFP was also applied at 1/100- 1/300 dilution into Blockaid+0.3% Triton-X 100 overnight at 4° C. or for 3 hours at room temperature.

### REFERENCES

Gerdes, M. J., Sevinsky, C. J., Sood, A., Adak, S., Bello, M. O., Bordwell, A., . . . Ginty, F. (2013). Highly multiplexed single-cell analysis of formalin-fixed, paraffin-embedded cancer tissue. Proceedings of the National Academy of Sciences of the United States of America, 110(29), 11982-11987. doi: 10.1073/pnas.1300136110.

Holden, M. A., & Cremer, P. S. (2003). Light activated patterning of dye-labeled molecules on surfaces. Journal of the American Chemical Society, 125(27), 8074-8075. doi: 10.1021/ja035390e.

Hopkins, A. L. (2008). Network pharmacology: the next paradigm in drug discovery. Nature Chemical Biology, 4(11), 682-690. doi: 10.1038/nchembio.118.

Jayagopal, A., Stone, G. P., & Haselton, F. R. (2008). Light-guided surface engineering for biomedical applications. Bioconjugate Chemistry, 19(3), 792-796. doi: 10.1021/bc700406w

Jungmann, R., Avendano, M. S., Woehrstein, J. B., Dai, M. J., Shih, W. M., & Yin, P. (2014). Multiplexed 3D cellular super-resolution imaging with DNA-PAINT and Exchange-PAINT. Nature Methods, 11(3), 313-U292. doi: 10.1038/nmeth.2835

Micheva, K. D., & Smith, S. J. (2007). Array tomography: A new tool for Imaging the molecular architecture and ultrastructure of neural circuits. Neuron, 55(1), 25-36. doi: 10.1016/j.neuron.2007.06.014.

Nelson, D. A., Manhardt, C., Kamath, V., Sui, Y. X., Santamaria-Pang, A., Can, A., . . . Larsen, M. (2013). Quantitative single cell analysis of cell population dynamics during submandibular salivary gland development and differentiation. Biology Open, 2(5), 439-447. doi: 10.1242/bio.20134309.

Schubert, W., Bonnekoh, B., Pommer, A. J., Philipsen, L., Bockelmann, R., Malykh, Y., . . . Dress, A. W. M. (2006). Analyzing proteome topology and function by automated multidimensional fluorescence microscopy. Nature Biotechnology, 24(10), 1270-1278. doi: 10.1038/nbt1250.

Abul-Husn, N. S., I. Bushlin, J. A. Moron, S. L. Jenkins, G. Dolios, R. Wang, R. Iyengar, A. Ma'ayan, and L. A. Devi. 2009. Systems approach to explore components and interactions in the presynapse. Proteomics 9:3303-3315.

Li, K. W., M. P. Hornshaw, R. C. Van der Schors, R. Watson, S. Tate, B. Casetta, C. R. Jimenez, Y. Gouwenberg, E. D. Gundelfinger, K. H. Smalla, and A. B. Smit. 2004. Proteomics analysis of rat brain postsynaptic density—Implications of the diverse protein functional groups for the integration of synaptic physiology. J. Biol. Chem. 279:987-1002.

Choudhary, J., and S. G. N. Grant. 2004. Proteomics in postgenomic neuroscience: the end of the beginning. Nat. Neurosci. 7:440-445.

Yoshimura, Y., Y. Yamauchi, T. Shinkawa, M. Taoka, H. Donai, N. Takahashi, T. Isobe, and T. Yamauchi. 2004. Molecular constituents of the postsynaptic density fraction revealed by proteomic analysis using multidimensional liquid chromatography-tandem mass spectrometry. J. Neurochem. 88:759-768.

Collins, M. O., H. Husi, L. Yu, J. M. Brandon, C. N. G. Anderson, W. P. Blackstock, J. S. Choudhary, and S. G. N. Grant. 2006. Molecular characterization and comparison of the components and multiprotein complexes in the postsynaptic proteome. J. Neurochem. 97:16-23.

Husi, H., M. A. Ward, J. S. Choudhary, W. P. Blackstock, and S. G. N. Grant. 2000. Proteomic analysis of NMDA receptor-adhesion protein signaling complexes. Nat. Neurosci. 3:661-669.

Pocklington, A. J., M. Cumiskey, J. D. Armstrong, and S. G. N. Grant. 2006. The proteomes of neurotransmitter receptor complexes form modular networks with distributed functionality underlying plasticity and behaviour. Mol. Syst. Biol. 2.

De Las Rivas, J., and C. Fontanillo. 2010. Protein-Protein Interactions Essentials: Key Concepts to Building and Analyzing Interactome Networks. Plos Computational Biology 6.

Hopkins, A. L. 2008. Network pharmacology: the next paradigm in drug discovery. Nat. Chem. Biol. 4:682-690.

Fredriksson, S., M. Gullberg, J. Jarvius, C. Olsson, K. Pietras, S. M. Gustafsdottir, A. Ostman, and U. Landegren. 2002. Protein detection using proximity-dependent DNA ligation assays. Nat. Biotechnol. 20:473-477.

Grant, D. M., W. Zhang, E. J. McGhee, T. D. Bunney, C. B. Talbot, S. Kumar, I. Munro, C. Dunsby, M. A. A. Neil, M. Katan, and P. M. W. French. 2008. Multiplexed FRET to Image Multiple Signaling Events in Live Cells. Biophys. J. 95:L69-L71.

Ramachandran, B., and J. U. Frey. 2009. Interfering with the Actin Network and Its Effect on Long-Term Potentiation and Synaptic Tagging in Hippocampal CA1 Neurons in Slices In Vitro. J. Neurosci. 29:12167-12173.

Fischer, M., S. Kaech, D. Knutti, and A. Matus. 1998. Rapid actin-based plasticity in dendritic spines. Neuron 20:847-854.

Fischer, M., S. Kaech, U. Wagner, H. Brinkhaus, and A. Matus. 2000. Glutamate receptors regulate actin-based plasticity in dendritic spines. Nat. Neurosci. 3:887-894.

Kim, C. H., and J. E. Lisman. 1999. A role of actin filament in synaptic transmission and long-term potentiation. J. Neurosci. 19:4314-4324.

Okamoto, K. I., T. Nagai, A. Miyawaki, and Y. Hayashi. 2004. Rapid and persistent modulation of actin dynamics regulates postsynaptic reorganization underlying bidirectional plasticity. Nat. Neurosci. 7:1104-1112.

Fukazawa, Y., Y. Saitoh, F. Ozawa, Y. Ohta, K. Mizuno, and K. Inokuchi. 2003. Hippocampal LTP is accompanied by enhanced F-actin content within the dendritic spine that is essential for late LTP maintenance in vivo. Neuron 38:447-460.

Micheva, K. D., B. Busse, N. C. Weiler, N. O'Rourke, and S. J. Smith. 2010. Single-Synapse Analysis of a Diverse Synapse Population: Proteomic Imaging Methods and Markers. Neuron 68:639-653.

Wahlby, C., F. Erlandsson, E. Bengtsson, and A. Zetterberg. 2002. Sequential immunofluorescence staining and image analysis for detection of large numbers of antigens in individual cell nuclei. Cytometry 47:32-41.

Gerdes, M. J., C. J. Sevinsky, A. Sood, S. Adak, M. O. Bello, A. Bordwell, A. Can, A. Corwin, S. Dinn, R. J. Filkins, D. Hollman, V. Kamath, S. Kaanumalle, K. Kenny, M. Larsen, M. Lazare, Q. Li, C. Lowes, C. C. McCulloch, E. McDonough, M. C. Montalto, Z. Y. Pang, J. Rittscher, A. Santamaria-Pang, B. D. Sarachan, M. L. Seel, A. Seppo, K. Shaikh, Y. X. Sui, J. Y. Zhang, and F. Ginty. 2013. Highly multiplexed single-cell analysis of formalin-fixed, paraffin-embedded cancer tissue. Proc. Natl. Acad. Sci. U. S. A. 110:11982-11987.

Brown, J. K., A. D. Pemberton, S. H. Wright, and H. R. P. Miller. 2004. Primary antibody-Fab fragment complexes: A flexible alternative to traditional direct and indirect Immunolabeling techniques. J. Histochem. Cytochem. 52:1219-1230.

Liu, H. C., and K. May. 2012. Disulfide bond structures of IgG molecules Structural variations, chemical modifications and possible impacts to stability and biological function. mAbs 4:17-23.

Lin, Y. C., and A. J. Koleske. 2010. Mechanisms of Synapse and Dendrite Maintenance and Their Disruption in Psychiatric and Neurodegenerative Disorders. In Annual Review of Neuroscience, Vol 33. S. E. Hyman, editor. Annual Reviews, Palo Alto. 349-378.

Sun, Y. S., R. N. Day, and A. Periasamy. 2011. Investigating protein-protein interactions in living cells using fluorescence lifetime imaging microscopy. Nat. Protoc. 6:1324-1340.

Lakowicz, J. R. 1999. Principles of Fluorescence Spectroscopy. Springer, New York.

Hermanson, G. T. 2013. Bioconjugate Techniques. Academic Press.

Nakagawa, Y., Capetill. S, and Jirgenso. B. 1972. Effect of chemical modification of lysine residues on confomation of human immunoglobulin-G. J. Biol. Chem. 247:5703-&.

Berlier, J. E., A. Rothe, G. Buller, J. Bradford, D. R. Gray, B. J. Filanoski, W. G. Telford, S. Yue, J. X. Liu, C. Y. Cheung, W. Chang, J. D. Hirsch, J. M. Beechem, and R. P. Haugland. 2003. Quantitative comparison of long-wavelength Alexa Fluor dyes to Cy dyes: Fluorescence of the dyes and their bioconjugates. J. Histochem. Cytochem. 51:1699-1712.

Wu, P. G., and L. Brand. 1994. Resonance energy transfer—methods and applications. Anal. Biochem. 218:1-13.

Podgorski, K., E. Terpetschnig, O. P. Klochko, O. M. Obukhova, and K. Haas. 2012. Ultra-Bright and -Stable Red and Near-Infrared Squaraine Fluorophores for In Vivo Two-Photon Imaging. PLoS One 7.

Longin, A., C. Souchier, M. Ffrench, and P. A. Bryon. 1993. Comparison of anti-fading agenst used in fluorescence microscopy—image-analysis and laser confocal microscopy study. J. Histochem. Cytochem. 41:1833-1840.

Yun, C. S., A. Javier, T. Jennings, M. Fisher, S. Hira, S. Peterson, B. Hopkins, N. O. Reich, and G. F. Strouse. 2005. Nanometal surface energy transfer in optical rulers, breaking the FRET barrier. J. Am. Chem. Soc. 127:3115-3119.

Cingolani, L. A., and Y. Goda. 2008. Actin in action: the interplay between the actin cytoskeleton and synaptic efficacy. Nat. Rev. Neurosci. 9:344-356.

Hotulainen, P., and C. C. Hoogenraad. 2010. Actin in dendritic spines: connecting dynamics to function. J. Cell Biol. 189:619-629.

What is claimed is:

1. A selectively cleavable probe comprising an F(ab) fragment linked to one or more labels by a chemically cleavable disulfide bond.

2. The selectively cleavable probe of claim 1, wherein the label is a fluorescent label.

3. The selectively cleavable probe of claim 1, wherein the label is a fluorescent protein.

4. The selectively cleavable probe of any one of claims 1-3, wherein the F(ab) fragment is a monovalent F(ab) fragment from a secondary IgG antibody.

5. The selectively cleavable probe of any one of claims 1-4, wherein the F(ab) fragment is an anti-Rabbit F(ab) fragment, anti-Mouse F(ab) fragment or another species specific F(ab) fragment.

6. The selectively cleavable probe of any one of claims 1-4, wherein the F(ab) fragment is an anti-biotin F(ab) fragment.

7. The selectively cleavable probe of any one of claims 3-6, wherein the fluorescent protein is selected from the group consisting of a Green Fluorescent Protein (GFP), Yellow Fluorescent Protein (YFP), Cyan Fluorescent Protein (CFP), and mCherry.

8. The selectively cleavable probe of any one of claims 3-6, wherein the fluorescent protein is a Green Fluorescent Protein (GFP) or a similar protein.

9. The selectively cleavable probe of any one of claims 3-6, wherein the fluorescent protein is a Green Fluorescent Protein (GFP).

10. The selectively cleavable probe of any one of claims 3-6, wherein the fluorescent protein is a fluorescent phycobiliprotein.

11. The selectively cleavable probe of claim 10, wherein the fluorescent phycobiliprotein is allophycocyanin, phycocyanin, phycoerythrin, or phycoerythrocyanin.

12. The selectively cleavable probe of any one of claims 3-11, wherein the fluorescent protein comprises a small fluorescent group surrounded by an inert shell that prevents the fluorophore from coming in contact with its surroundings.

13. The selectively cleavable probe of any one of claims 3-12, wherein the fluorescent protein comprises a small fluorescent group surrounded by proteins.

14. The selectively cleavable probe of any one of claims 3-13, wherein the fluorescent protein comprises a small fluorescent group surrounded by a protein beta barrel.

15. The selectively cleavable probe of any one of claims 1-14, wherein the label does not form a non-cleavable bond with its surroundings upon excitation with light.

16. The selectively cleavable probe of any one of claims 1-15, wherein the label is not a small-molecule fluorophore.

17. A process of immunostaining a sample comprising staining the sample with the selectively cleavable probe of any one of claims 1-16.

18. A process of immunostaining a sample comprising
a) obtaining a probe comprising a primary antibody against a preselected target linked to biotin,
b) staining the sample with the probe of step a),
c) staining the sample with the selectively cleavable probe of any one of claims 7-16, wherein the F(ab) fragment is an anti-biotin F(ab) fragment.

19. The process of claim 18, wherein the primary antibody against the preselected target is conjugated to biotin or linked to biotin by a disulfide bond.

20. The process of claim 18, wherein the primary antibody against the preselected target is linked to biotin by a disulfide bond.

21. The process of any one of claims 18-20, further comprising washing off any unbound primary antibody after step b).

22. The process of any one of claims 17-21 further comprising imaging the sample after staining the sample with the selectively cleavable probe.

23. The process of any one of claims 17-22, further comprising destaining the sample by cleaving the bond between the F(ab) fragment and the one or more labels.

24. The process of claim 23, wherein the destaining comprises contacting the sample with a reducing agent.

25. The process of claim 24, wherein the amount of the reducing agent is 5-50 mM or 20-50 mM.

26. The process of claim 24 or 25, wherein the reducing agent is a disulfide reducing agent.

27. The process of any one of claims 24-26, wherein the reducing agent is tris(2-carboxyethyl)phosphine (TCEP) or Dithiothreitol (DTT).

28. The process of any one of claims 23-27, wherein the destaining is performed under near physiological conditions or under mild conditions.

29. The process of any one of claims 18-28 further comprising cleaving unbound biotin moieties on the primary antibody.

30. The process of any one of claims 17-29 further comprising the step of staining the sample with a permanent label that is unaffected by disulfide reducing agents.

31. The process of claim 30, wherein the permanent label is phalloidin-Alexa 647.

32. The process of any one of claims 30-31, wherein the permanent stain is not affected by the destaining.

33. The process of any one of claims 18-32, wherein the target is selected from the group consisting of a protein, and an antigen.

34. The process of any one of claims 18-33 further comprising repeating the process for a second preselected target.

35. The process of any one of claims 18-34, further comprising repeating the process for a third preselected target.

36. The process of any one of claims 18-35, further comprising repeating the process for a fourth preselected target.

37. The process of any one of claims 18-36, further comprising repeating the process for a fifth preselected target.

38. The process of any one of claims 18-37, further comprising repeating the process 6 or more times for additional preselected targets.

39. The process of any one of claims 17-38, wherein the sample is not degraded during the process.
